(19) United States
(12) Patent Application Publication
(10) Pub. No.: US 2022/0015992 A1
(43) Pub. Date: Jan. 20, 2022

(54) TOPICAL COMPOSITION USING A TWO-PART FORM FACTOR

(71) Applicant: EXPONENT BEAUTY, INC., San Francisco, CA (US)

(72) Inventors: Elizabeth Robinson Whitman, San Francisco, CA (US); Nancy W. Leung, Oakland, CA (US)

(21) Appl. No.: 17/375,588

(22) Filed: Jul. 14, 2021

Related U.S. Application Data
(60) Provisional application No. 63/052,497, filed on Jul. 16, 2020.

(51) Int. Cl. A61K 8/02; A61K 9/00; A61K 9/06; A61K 9/14; A61K 45/06; A61Q 19/00; A61Q 19/08

(52) U.S. Cl. CPC A61K 8/022; A61K 8/0204; A61K 9/0014; A61K 9/06; A61K 9/14; A61K 2800/805; A61Q 19/007; A61Q 19/08; A61K 2800/524; A61K 2800/882; A61K 45/06

(57) ABSTRACT

According to some embodiments, a topical composition and method of forming the same are provided, comprising a powder complex including an active component, wherein the powder complex has a pre-defined first dosage; and a universal activator serum having a pre-defined second dosage, wherein the combination of the powder complex and the universal activator serum generates the topical composition having a third pre-defined dosage and a viscosity of 10-30K centipoise (CPS). Numerous other aspects are provided.

### CROSS REFERENCE TO RELATED APPLICATION

The present application claims priority from the following U.S. Provisional Patent Application, which is hereby incorporated by reference herein in its entirety for all purposes: U.S. Provisional Patent Application Ser. No. 63/052,497, filed Jul. 16, 2020, and entitled “TOPICAL COMPOSITION USING A TWO-PART FORM FACTOR” (Attorney Docket No. E10.002P).

### BACKGROUND

Antioxidants are substances that inhibit oxidation, thereby preventing or slowing damage to cells caused by free radicals, which are produced by oxidation. Free radicals are unstable molecules that the body produces as a reaction to environmental and other pressures. When free radicals accumulate in the body, they may cause a state known as oxidative stress. Antioxidants may be referred to as “free-radical scavengers” as they may scavenge free radicals from the body cells and prevent or reduce the damage caused by oxidation. Antioxidants may be used in topical formulations as skin treatments. In this role they may provide protection to the skin from the ravages of free radicals.

However, the antioxidants may also be susceptible to damage from external sources. For example, antioxidants may be susceptible to degradation by exposure to heat or light, contact with oxygen or moisture. This degradation may result in discoloration of the skin treatment in which they are contained, as well as diminished efficacy when applied to the skin. Conventional topical skin treatments containing antioxidants are not likely to deliver their maximum potency and are not likely to maintain their potency.

It would be desirable to provide a topical composition that improves and maintains the amount of antioxidant delivered to the skin.

### SUMMARY

According to some embodiments, a topical composition is provided including a powder complex including an active component, wherein the powder complex has a pre-defined first dosage; and a universal activator serum having a pre-defined second dosage, wherein the combination of the powder complex and the universal activator serum generates the topical composition having a third pre-defined dosage and a viscosity of 10-30K centipoise (CPS).

According so some embodiments, a method of forming a topical composition is provided including providing a source of a powder complex including an active component; providing a source of a universal activator serum; dispensing a predefined first dosage of the powder complex onto a surface; dispensing a pre-defined second dosage of the universal activator serum onto the surface; combining the dispensed powder complex with the dispensed universal activator serum to form the topical composition having a third pre-defined dosage and a viscosity of 10-30K centipoise (CPS).

A technical effect of some embodiments of the invention is an improved topical composition including active ingredients that have not been degraded before point of sale, and may not be degraded during the period of use. In embodiments, the topical composition provides a greater stability of biological activity as well as compositional integrity than conventional antioxidant topical compositions. Embodiments provide a composition that is particularly useful in methods for scavenging free radicals on the skin, and treating and reducing the symptoms of skin conditions, including but not limited to, fine lines and wrinkles, deep wrinkles, pitting and bumps, increased pore size, keratoses, skin flakiness or roughness, unevenness or blotching of skin tone, yellowing of the skin, redness and rosacea, dark undereye shadows or circles, loss of skin elasticity, sagging (including puffiness in the eye area and jowls), elastosis, loss of skin firmness or tightness, hyperpigmentation, age spots and freckles, abnormal differentiation, collagen breakdown, spider veins, etc. Embodiments provide a method of reducing or preventing free radical damage which comprises applying to cells a free radical scavenging-effective amount of at least one antioxidant or other suitable active ingredient. Embodiments provide a process that encompasses the treatment or prevention of undesirable skin conditions in which a contributory factor is the adverse effect of free radicals. Embodiments provide a method of generating a topical composition immediately prior to topical application by a user.

With this and other advantages and features that will become hereinafter apparent, a more complete understanding of the nature of the invention can be obtained by referring to the following detailed description.

### BRIEF DESCRIPTION OF THE DRAWINGS

FIG. 1 illustrates a block diagram of a topical composition according to some embodiments.

FIG. 2 illustrates a table of components for different powder complexes according to some embodiments.

FIG. 3 illustrates a flow diagram of a process for forming the topical composition according to some embodiments.

### DETAILED DESCRIPTION

As described above, active ingredients that have been clinically proven to have positive performance on skin are often unstable, degrading rapidly when in contact with oxygen, UV and other light sources. A non-exhaustive example of an active ingredient is an antioxidant. Antioxidants include, but are not limited to, Vitamin C, Retinol (pure Vitamin A), CoQ10 (Conenzyme Q-10—Ubiquinone), Resveratrol, Green Tea, etc. Other non-exhaustive examples of active ingredient are Willow Bark extract and Enzymes including Papain and Bromelian, etc. Willow Bark extract may be sensitive to water (e.g., it may recrystallize) and light (it may break-down), and Enzymes may be sensitive to water (e.g., degrade easily in water), as are other antioxidants. While conventional skincare products may include these active ingredients, an analysis of these conventional products indicates that the product may lose 50% of its concentration of the active ingredient in eight weeks of use. Additionally, most conventional skincare products on the market are designed to maximize the shelf life of a finished product (typically targeting 2-3 years before), rather than the stability and efficacy of the active ingredients. As such, a user may receive a product that is nearly two years old before even opening the container.

Some conventional skincare products provide for a user to add an antioxidant (e.g., vitamin C or vitamin A) to an already existing moisturizer or serum, and that moisturizer/serum may then deliver the antioxidant to the skin. However, the antioxidant may not be easy to work with. For example, a user should avoid breathing in vitamin C (L ascorbic acid) powder in its pure form. Additionally, these instances rely on the user adding an indiscriminate amount of antioxidant to an indiscriminate amount of moisturizer/serum and do not rely on a perfect dose or ratio of antioxidant to moisturizer/serum to efficiently deliver an optimally effective amount of the antioxidant to the targeted skin.

One or more embodiments provide a topical composition that is “made in the moment” by a consumer, focusing on maximizing the active ingredients and efficacy of those ingredients, rather than shelf life. Embodiments provide for a series of “freshly-made” and highly concentrated formulas that keep the active ingredients at their most stable and optimal form until combined on a per-use basis. Instead of maximizing shelf life with stabilizers, emulsifiers and other ingredients that primarily benefit formula stability, the topical composition of one or more embodiments is focused on fresh formulas that primarily benefit the skin and deliver positive performance.

Embodiments provide a topical composition that combines an active concentrate/powder complex and hydrating liquid base (two-part form factor) that are separated until combined immediately prior to application, to ensure the powerful ingredients remain their most potent and active until use. Embodiments provide for the consumer to mix a pre-defined dosage of a powder complex, including clinically active ingredients, with a pre-defined dosage of a universal liquid activator serum immediately prior to application to generate an “instant superior serum” with an accurate, effective concentration level of actives, which is a topical composition that targets a specific skin concern. The topical composition may have the feeling and appearance of a creamy serum, cream or gel-cream, which is texturally different from either the powder complex or the transparent universal activator serum. It is noted that by maintaining the active ingredients in a powder form in an enclosed glass container (or other suitable container) until they are mixed by the consumer, the active ingredients are kept stable (e.g., unreactive to oxygen, water and light), meaning they maintain their stated concentration and have increased shelf life. As used herein, “active ingredients” may refer to those ingredients having a correction action to them and/or some efficacy towards the application location (e.g., skin).

It is noted that a “serum” is typically a highly concentrated thin liquid that may deliver a high concentration of active ingredients; and a “cream” is a moisturizer including at least one of a humectant (to draw in moisture) and moisturizing agent (to trap moisture). While a serum may add moisture, it typically does not trap moisture. The “superior serum” of one or more embodiments may be thicker than a conventional serum (e.g., water-based serum) and may have a gel-like texture that is able to trap moisture therein.

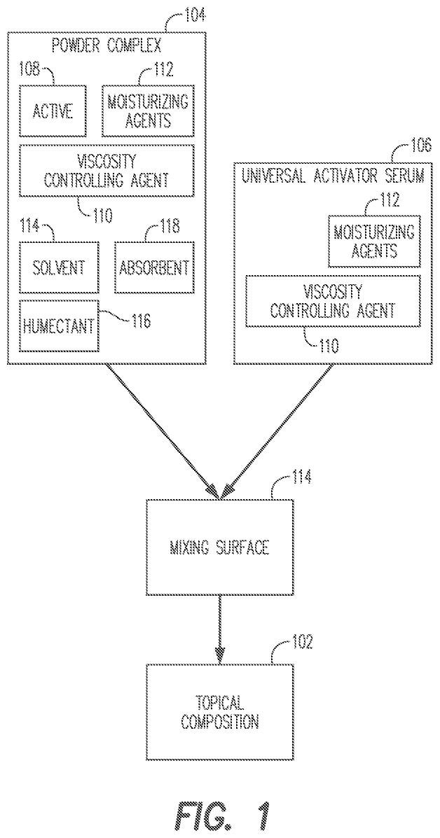

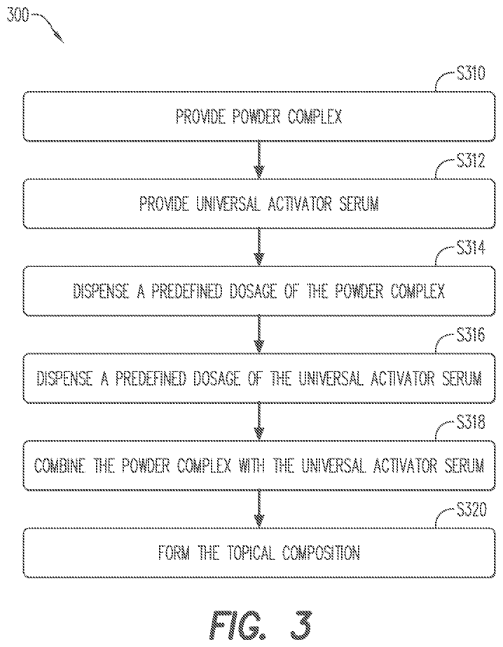

Turning to FIGS. 1-3, a topical composition 102 is formed via process 300 from the combination of an active concentrate/powder complex 104 and a universal activator serum 106. It is noted that the terms “universal activator serum”, “universal accelerator serum” and “universal hydrator serum” may be used interchangeably. The powder complex 104 may be formed, in part, from an active component 108 (e.g., antioxidant and/or other active), one or more viscosity controlling agents 110, one or more moisturizing agents 112, and one or more absorbents. The powder complex 104 may also include at least one other ingredient. The other ingredients may be at least one of: one or more solvents, one or more humectants, one or more absorbents, one or more skin conditioning agents, one or more pigments/opacifiers, one or more aromatic agents, one or more preservatives and one or more pH adjustor agents. The universal activator serum 106 may be formed, in part, from an active ingredient 108 (e.g., Hyaluronic Acid and derivatives), one or more viscosity controlling agents 110 (e.g., Cellulose Gum), and one or more moisturizing agents 112 (e.g., PEG-8 Dimethicone). The universal activator serum may also include at least other ingredient. The other ingredients may be at last one of: one or more solvents (e.g., Water, Dimethyl Isosorbide), one or more humectants (e.g., Propanediol, Glycerin, Betaine, 1,2-Hexanediol, Sodium Acetylated Hyaluronate, Sodium Hyaluronate, Sodium Hyaluronate Crosspolymer, Hydrolized Sodium Hyaluronate, Pentylene glycol), and one or more skin conditioning agents (e.g., PEG-8 Dimethicone, Sodium Acetylated Hyaluronate, Sodium Hyaluronate, Sodium Hyaluronate Crosspolymer, Hydrolized Sodium Hyaluronate, Panthenol, Pantolactone, and Ethylhexylglycerin).

Initially, at S310, a powder complex source is provided (including the active component). Then at S312, a universal activator serum is provided. In one or more embodiments, the topical composition 102 (mixture of the powder complex 104, including the active component 108, and the universal activator serum 106) may be formed by dispensing a predefined first dosage of the powder complex 104 onto a mixing surface 114 at S314. The mixing surface 114 may be skin, a receptacle, or any other suitable surface. Next, at S316, a predefined second dosage of the universal activator serum 106 may be dispensed onto the mixing surface 114. It is noted that while the predefined dosage of each of the powder complex 104 and the universal activator serum 106 may vary based at least on the active component 108 of the powder complex 104, the predefined dosage of the powder complex 104 may be in a range of 0.1 to 0.25 grams, preferably 0.2 grams and the predefined dosage of the universal activator serum 106 may be in a range of 0.2 to 0.5 grams, preferably 0.4 grams. In embodiments, the predefined dosage of each of the powder complex 104 and the universal activator serum 106 may be the same or different. In embodiments, the ratio of powder complex 104 and the universal activator serum 106 in the topical composition 102 may be one part powder complex to two parts universal activator serum 106. Other suitable ratios may be used.

After both the predefined dosages of the powder complex 104 and the universal activator serum 106 are dispensed on the mixing surface 114 at S316, they are combined by mixing them continuously for a time of 10 (ten) seconds or less at S318, (e.g., for five to ten seconds). The mixing may at least one of dissolve and suspend the powder complex 104 in the universal activator serum 106 and forms the topical composition 102 at S320. In one or more embodiments, the active component may be between 0.25 and 10% of the topical composition 102. The topical composition may have a pH of 3.5-7.0.

Topical compositions 102 of the present invention are a single-phase solution composition resulting from a 2-part composition. “Single-phase solution compositions” means herein that the composition has one phase, that of a superior serum phase, and is visually and texturally homogenous, with no perceptible particulate material.

In one or more embodiments, the topical composition 102 may have a texture of a luxurious medium-weight “superior serum”. The topical composition 102 may have a viscosity of 10-30K centipoise (CPS). During application to the skin, the topical composition spread easily on the skin with a fresh, lightweight, hydrating sensation but with moderate cushion and slight lubricity and a velvety feel. Application of the topical composition 102 may feel hydrating and moisturizing, with no tacky, oily, slick feel. The topical composition 102 may absorb quickly (in 10 to 30 seconds) once applied, and does not pill, drag or soap during application. After application, the skin may feel moisturized, hydrated, smooth and flexible.

The topical compositions 102 of one or more embodiments provide for the active component 108 contained therein to be stable and not readily degraded, thereby retaining more activity between the time of formulation (mixing in the moment) and the time of application, thus providing a more effective delivery of that active component.

An additional unexpected advantage of the topical composition 102 of one or more embodiments is the extended shelf life while maintaining potency of the active component 108.

The topical composition 102 may be used for the production of cosmetic preparations, or dermatological preparations, more particularly topical treatment preparations. Topical application to a surface may be a surface such as the skin, for example. Non-exhaustive examples of topical compositions and their respective benefits include: a Retinol topical composition 102 that may provide correction of visible skin aging, reduce appearance of fine lines and wrinkles; a CoQ10 topical composition 102 that may provide prevention of visible skin aging and help to prevent the appearance of fine lines and wrinkles; a botanical antioxidant topical composition 102 that may provide soothing and address sensitive skin and redness; a Vitamin C topical composition 102 that may brighten the skin and address sun damage and dullness; and a probiotic enzyme topical composition 102 that may provide skin clearing in that it may address blemishes, pore size and oily skin.

Powder Complex

In one or more embodiments, the powder complex 104 may be a non-dusty, slightly granular, free-flowing powder that is able to dissolve and thicken immediately upon contact with liquid. The powder complex 104 may remain free-flowing over time (e.g., does not cake/harden). It is noted that including the active components 108 in the powder complex 104 may help support the efficacy of the active component 108 by preserving its stability. The powder complex may have an approximate density in a range of +/−10% of 0.51 g/ml.

Active Component

The topical compositions of one or more embodiments utilize antioxidants and other actives (e.g., Willow Bark extract and Probiotics) as an active component 108 in the powder complex 104. The active components 108 employed in the topical composition 102 may be any that are cosmetically or pharmaceutically acceptable for topical application to skin. By “cosmetically or pharmaceutically acceptable” is meant compounds that may be used in safe and effective amounts on skin, hair or nails, preferably on human skin, hair or nails. The active components 108 used in the topical compositions 102 described by one or more embodiments may be understood to encompass naturally occurring active components, synthetic derivatives of active components, as well as plant extracts containing at least one active component. Examples of active components used in the topical compositions include, but are not limited to, Vitamin C (Ascorbic acid), Quercetin, Epigallocatechin gallate (e.g., key antioxidant in Green Tea), Retinol/Vitamin A, CoQ10(Coenzyme Q-10—Ubiquinone), Resveratrol, Tocopheryl Acetate (form of Vitamin E), Green Tea EGCG, Enzymes, Willow Bark Extract and Probiotics).

Viscosity Controlling Agent

In one or more embodiments, the powder complex may include one or more viscosity controlling agents 110 (“gelling agents”) in powder form. The topical composition 102 may be formed from a viscosity controlling system, not an emulsion. Emulsions may be difficult for a user to generate on their own and may detract from the active component concentration. Viscosity controlling agents 110, on the other hand, may provide a thickness and body that suspends the active component (and other powdered moisturizing agents) in the topical composition 102. A non-exhaustive example of a viscosity controlling agent is hydroxyethyl acrylate/sodium acryloyldimethyl taurate copolymer and distartch phosphate. Other suitable viscosity controlling agents may be used. It is noted that the addition of this non-exhaustive example of viscosity controlling agent may contribute to the instant (i.e., 10 (ten) seconds or less) formation of a nice smooth “superior serum” (i.e., topical composition 102) when the powder complex 104 including this viscosity controlling agent 110 is mixed with the universal activator serum 106. Other viscosity controlling agents may similarly contribute to the formation of the topical composition in 10 (ten) seconds or less. While other viscosity controlling agents, besides hydroxyethyl acrylate/sodium acryloyldimethyl taurate copolymer may form gels, they may result in an inconsistent (not smooth) texture and/or may take more than ten seconds to form the superior serum consistency. The inventors note that the instant formation of the nice smooth “superior serum” consistency is desirable as a user is creating the topical composition in their hand or a small container/receptacle at a time immediately prior to application to the skin. The viscosity controlling agent 110 may provide a slip effect that improves the feeling on the skin, as well as may assist in keeping moisture on the skin and in improving absorption of the topical composition.

Moisturizing Agents

In one or more embodiments, the powder complex 104 may include one or more moisturizing agents 112 in powdered form. The moisturizing agent 112 may be a powdered emollient to soften, smooth and/or add flexibility to the skin. Non-exhaustive examples of powdered emollients include, but are not limited to, dimethicone, dimethicone crosspolymer, and shea butter (Butyrospermum parkii). The powdered moisturizing agent 112 may result in the topical composition 102 having the properties of a moisturizer (e.g., soften skin, smooth skin, add suppleness, lock in moisture, prevent moisture loss) including the texture of a moisturizer. The moisturizing agent 112 may enhance the ability of the topical composition 102 to instantly form when the powder complex 104 is mixed with the universal activator serum 106. It is noted that the moisturizing agent 112 may also provide moisture/emollience to the skin and lubricity to the mixed formula.

Humectant

In some embodiments, the powder complex may include one or more humectants 116. The humectant 116 may attract and add moisture to skin when the powder complex is mixed with the activator. Non-exhaustive examples of humectants 116 include, but are not limited to, Betaine, and Aloe Barbadensis Leaf Juice.

Absorbent

The powder complex 104, in one or more embodiments, may include an absorbent 118. The absorbent 118 may keep the powder free-flowing and uniform, keep ingredients from interacting prior to mixing with the activator, and help stabilize active ingredients by absorbing excess water or humidity. Non-exhaustive examples of absorbents include, but are not limited to, silica, calcium silicate, distarch phosphate and maltodextrin. It is noted that some absorbents may also provide skin conditioning properties (e.g., maltodextrin), as described further below.

Skin Conditioning

The powder complex may, in some embodiments, include one or more skin conditioning agents. The skin conditioning agent may help to improve the look and feel of skin. Non-exhaustive examples of skin conditioning agents include, but are not limited to, asiaticoside (a key soothing compound in Centella asiatica), Ethylhexylglycerin, Salix alba (Willow) Bark Extract (an astringent that may be a key clearing extract rich in salicylates), Bromelain (enzyme), Lactobacillus Ferment, Papain (enzyme), and Allantoin (protectant). It is noted that Salix alba (Willow) Bark Extract may also be an active.

Pigment/Opacifier

One or more pigments/opacifiers may be used in the powder complex 102 in embodiments to impart a color to the topical composition and impart an immediate visible brightness and smoothness to skin. Non-exhaustive examples of pigment include, but are not limited to, Mica, and Titanium Dioxide (CI 77891), where “CI” is Color Index Number.

Aromatic Agent

One or more aromatic agents may be used in the powder complex 104 in embodiments to impart a pleasant scent to the topical composition. Non-exhaustive examples of aromatic agents include, but are not limited to, Citrus nobilis (Mandarin Orange) Peel Oil, Ormenis multicaulis Oil, Anthemis nobilis (Roman Chamomile) Oil, Rosa damascena Flower Oil, Rose Geranium (Pelargonium graveolens) Oil, Rosmarinus officinalis (Rosemary) Leaf Oil and Lavandula angustifolia (Lavender) Oil.

Preservative

Some embodiments may include one or more preservatives in the powder complex to prevent the growth of bacteria, mold, and other microorganisms. Any preservative commonly used in cosmetic formulations may be an acceptable preservative for the powder complex. Non-exhaustive examples of preservatives include, but are not limited to, potassium sorbate and phenoxyethanol.

pH Adjustor Agent

Some embodiments may include one or more pH adjustor agents in the powder complex 104 to maintain the appropriate pH for skin. Non-exhaustive examples of pH adjustor agents include, but are not limited to, Citric Acid.

In one or more embodiments, when forming the powder complex 104, the ingredients may be mixed in a particular order. For example, oils may be processed with some absorbent powders that can hold oils and still allow for a free-flowing powder. In other embodiments, the ingredients may be mixed in a random order.

Universal Activator Serum

In one or more embodiments, the powder complex 104 including the active component 108 may be formulated into the topical composition 102 via combination with a topically acceptable carrier, referred to herein as the “universal activator serum” 106. The universal activator serum 106 may provide at least two functions. The first function is as a carrier for the powder complex 104, which includes the active component 108. The universal activator serum 106 may be lightweight and flowable enough to dispense from a pump with a dip tube, pourable container, dropper, or any other suitable dispenser. As a carrier, the universal activator serum 106 may be one that is pharmaceutically or cosmetically acceptable, that is, a vehicle for either pharmaceutical or cosmetic use, intended for application to external body surfaces, such as skin, hair or nails. The universal activator serum 106 delivers the active components 108 and other ingredients of the powder complex 104 to the intended target and may not cause harm to the average human or other recipient organisms when applied to the surface intended to be treated. As used herein, “pharmaceutical” or “cosmetic” will be understood to encompass both human and animal, preferably mammalian, pharmaceuticals or cosmetics, with which the active component is compatible. The second function is that the universal activator serum 106 may have hydration properties prior to combination with the powder complex 104. While the universal activator serum 106 described herein is a serum, other suitable carriers may be used, including but not limited to, a gel, a spray, water- or oil-based carrier, and the like.

A non-exhaustive example of the universal activator serum 106 is a hyaluronic liquid activator serum including solvents (e.g., water, dimethyl isosorbide) wetting agents and humectants (e.g., glycerin, propandiol, etc.), hyaluronic acid and derivatives (e.g., Sodium Hyaluronate, Sodium Acetylated Hyaluronate, Sodium Hyaluronate Crosspolymer, Hydrolyzed Sodium Hyaluronate), panthenol, etc. The wetting agents and humectants of the universal activator serum 106 may help form the topical composition 102 more quickly (as compared to not using them) when combined with the powder complex 104, as they may help dissolve the ingredients in the powder complex 104 more quickly by saturating (“wetting”) the ingredients, which helps them to dissolve faster and create the topical composition 102. In the dissolved state, the active ingredients 108 and all of the other components of the powder complex may be evenly distributed throughout the topical composition. The universal activator serum 106 may have a viscosity of 1 k-5 k CPS. Other suitable viscosity measurements may be used.

It is noted that while the universal activator serum 106 described herein is a “universal” carrier in that it may be used with any number of active components/powder complexes, the chemical composition of the carrier may vary according to the identity of the active component and the intended final use. Any suitable composition that is useful for topical delivery, for example, aqueous dispersions, anhydrous compositions in liquid, solid or powder form, emulsions (oil or silicone-in-water, water-in-oil or silicone, multiple emulsions, microemulsions, nano-emulsions) may be employed, provided the carrier is compatible with the active component and all of the other components of the powder complex. The carrier may be formulated for application to the skin, for example, skin care products (e.g., sunscreens, self-tanners, skin moisturizers and conditioners, exfoliators, anti-acne compositions, anti-aging compositions and the like and color cosmetics as well as hair care, etc.).

It is noted that at least one of the powder complex 104 and universal activator serum 106 may contain other topically useful ingredients which may enhance or complement the activity of the topical composition 102. Non-exhaustive examples of categories of topically useful and acceptable ingredients that may be used in the topical composition are: formulation aids such as anti-caking agents, anti-foaming agents, fillers and bulking agents, thickeners, gellants, structuring agents and emulsion stabilizers; surfactants and emulsifiers; agents to enhance adhesion and retention on the intended target; propellants; biogenic agents, UV protection factors, pearlizing waxes, silicone compounds, fats, waxes, fats, deodorants, antiperspirants, antidandruff agents, film formers, insect repellents, self-tanning agents, and neutralizing agents.

The present invention provides a method for regulating the condition of skin tissue, which comprises applying to the tissue a topical composition containing an effective amount of an active component. An effective amount of an active component is an amount of the active component capable of achieving the therapeutic effect intended. This will typically be in the range of the amount used for a given active ingredient.

As used herein, the term “regulating” skin condition means both the treatment and prevention of skin conditions, in particular, those conditions that represent a pathology, as well as those that may cause discomfort, or present an unattractive or less appealing appearance. Non-exhaustive examples of skin benefits resulting from the application of the composition described by embodiments include, but are not limited to, brightening, visible aging skin prevention, visible aging skin correction, firming, brightening, calming, and clearing.

It is noted that the skin may be affected by a number of internal factors, as well as external environmental factors such as sun, pollution, or cigarette smoke. Embodiments treat skin conditions resulting from these factors. The skin conditions include, but are not limited to, fine lines and wrinkles, deep wrinkles, pitting and bumps, increased pore size, keratoses, skin flakiness or roughness, unevenness or blotching of skin tone, yellowing of the skin, redness and rosacea, dark undereye shadows or circles, loss of skin elasticity, sagging (including puffiness in the eye area and jowls), elastosis, loss of skin firmness or tightness, hyperpigmentation, age spots and freckles, abnormal differentiation, collagen breakdown, spider veins, etc.

The methods of application of the topical composition may vary depending upon the identity of the active component and on the intended end use of the topical composition. In general, for the regulation of various skin conditions, or for the prevention or treatment of conditions, topical compositions may be applied either in advance of the exposure to the potentially damaging factor (e.g., sunlight), or after the exposure or the amelioration of damage that may have occurred as a result of the exposure. Application may continue for as long as the exposure to the noxious stimulus continues, or may be discontinued when relief is obtained. When being used to treat, improve or prevent a more permanent condition, for example, everyday exposure to sun or environmental damage, or for the treatment or prevention of signs associated with aging, the topical composition is preferably applied chronically, to prevent the occurrence or recurrence of the condition. By “chronic” application, it is meant herein that the period of topical application may be over the lifetime of the user, and any suitable amount of time within that lifetime (e.g., one month, three months, one year, five years, etc.), thereby resulting in the treatment, prevention or reduction of the condition in question.

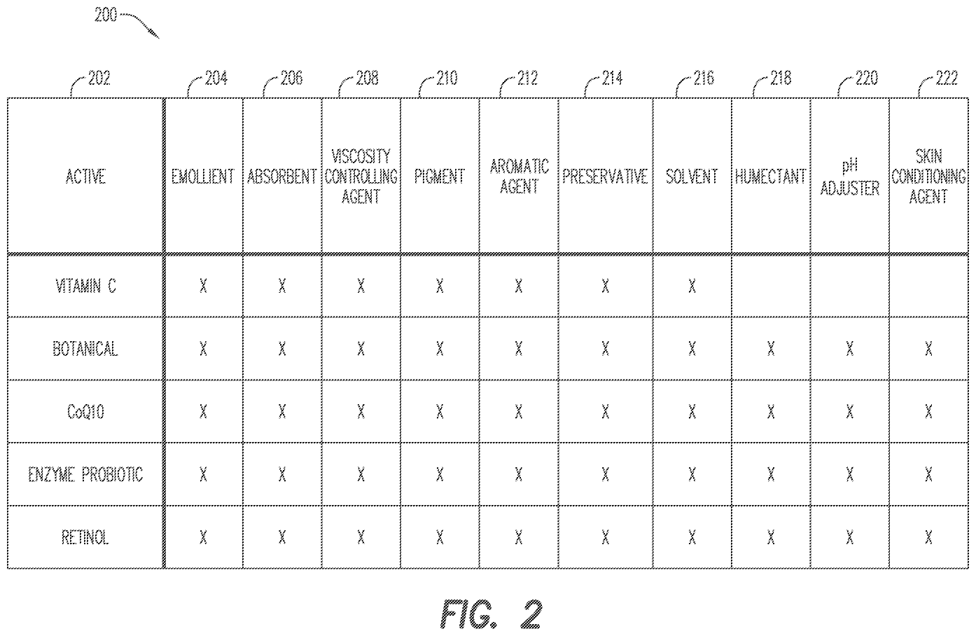

FIG. 2 provides a table 200 of powder complexes, listing a plurality of active components 202 (e.g., Vitamin C, Botanical extract, CoQ10, Enzyme Probiotic, Retinol), and whether the powder complex that includes the given active component also includes at least one of a moisturizing agent, 204, an absorbent 206, a viscosity controlling agent 208, a pigment 210, an aromatic agent 212, a preservative 214, a solvent 216, a humectant 218, a pH Adjustor 220 and a skin conditioning agent 222. Inclusion of the component is indicated by an “X” and exclusion of the component is indicated by an empty space.

The topical compositions of embodiments are further illustrated by the following non-limiting examples. It is noted that the ratio of powder complex to activator may be 1:2 for all of the following examples, although other suitable ratios may be used.

### EXAMPLES

### Example 1—Retinol

In this example, the retinol may be 0.25% of the finished topical composition (powder complex plus universal activator) in a per post-mixed use, and may include soothing agents. In other examples, the retinol may be a different (e.g., higher or lower) percentage of the finished topical composition. The soothing agents may include, but are not limited to Vitamin E, allantoin, which may offset potential irritation of retinol, as well as powdered moisturizing agents and lavender essential oil.

The ingredients may include betaine, dimethicone crosspolymer, Glycine soja (soybean) oil, silica, hydroxyethyl acrylate/sodium acryloyldimethyl taurate copolymer, allantoin, calcium silicate, distarch phosphate, water, Butyrospermum parkii (shea) butter, Lavandula angustifolia (lavender) oil, mica, potassium sorbate, quercetin, retinol, titanium dioxide, citric acid, dimethicone, tocopheryl acetate, ethylhexylglycerin and phenoxyethanol. Other suitable ingredients may be used.

### Example 2—CoQ10

In this example, the CoQ10 may be 0.5-1% per post-mixed use, and the topical composition may include powdered moisturizing agents and rose essential oil. In other examples, the CoQ10 may be a different (e.g., higher or lower) percentage of the finished topical composition.

The ingredients may include betaine, distarch phosphate, silica, hydroxyethyl acrylate/sodium acryloyldimethyl taurate copolymer, dimethicone, calcium silicate, ubiquinone, water, Butyrospermum parkii (shea) butter, mica, potassium sorbate, quercetin, titanium dioxide, dimethicone crosspolymer, distarch phosphate, citric acid, ethylhexylglycerin, phenoxyethanol, and Rose Geranium (Pelargonium graveolens) Flower Oil. Other suitable ingredients may be used.

### Example 3—Botanical Extract

The botantical powder complex may include an active blend of green tea ECGC, resveratrol, Centella asiatica asiaticoside. In this example, the botanical blend may be 1% of the finished topical composition in a per post-mixed use, and the topical composition may include aloe powder, powdered moisturizing agents and chamomile essential oil. In other examples, the botanical blend may be a different (e.g., higher or lower) percentage of the finished topical composition.

The ingredients may include betaine, distarch phosphate, silica, hydroxyethyl acrylate/sodium acryloyldimethyl taurate copolymer, dimethicone, epigallocatechin gallate, calcium silicate, water, buytrospermum parkii (shea) butter, aloe barbadensis leaf juice, asiaticoside, mica, potassium sorbate, quercetin, resveratrol, titanium dioxide, dimethicone crosspollymer, citric acid, ethylhexylglycerin, Anthemis nobilis (Roman Chamomile) oil, and phenoxyethanol. Other suitable ingredients may be used.

### Example 4—Vitamin C

The Vitamin C powder complex may include L Ascorbic Acid and be 10% of the finished topical composition in a per post-mixed use. In other examples, the Vitamin C powder complex may be a different (e.g., higher or lower) percentage of the finished topical composition. The topical composition may include powdered moisturizing agents and lemon essential oil.

The ingredients may include ascorbic acid, dimethicone crosspolymer, silica, dimethicone, hydroxyethyl acrylate/sodium acryloyldimethyl taurate copolymer, calcium silicate, maltodextrin, Butyrospermum parkii (shea) butter, Citrus nobilis (mandarin orange) peel oil, distarch phosphate, mica, potassium sorbate, quercetin, and titanium dioxide. Other suitable ingredients may be used.

### Example 5—Clearing Enzyme Probiotic

The Enzyme Probiotic Clearing powder may include an active blend of Papain, Lactobacillus Ferment, Bromelain, and Willowbark extract. In this example the active blend may be 1.7% of the finished topical composition in a per post-mixed use, and the topical composition may include other clearing elements, as well as rosemary essential oil. In other examples, the active blend may be a different (e.g., higher or lower) percentage of the finished topical composition.

The ingredients may include betaine, distartch phosphate, silica, hydroxyethyl acrylate/sodium acryloyldimethyl taurate copolymer, dimethicone, Salix alba (willow) bark extract, calcium silicate, maltodextrin, water, Butyrospermum parkii (shea) butter, bromelain, lactobacillus ferment, mica, papain, potassium sorbate, quercetin, Rosmarinus officinalis (rosemary) leaf oil, titanium dioxide, dimethione crosspolymer, citric acid, ethylhexylglycerin, and phenoxyethanol. Other suitable ingredients may be used.

### Example 6—Hyaluronic (Universal) Activator Serum

The ingredients may include water, propanediol, glycerin, betaine, 1,2-hexanediol, cellulose gum, ethylhexylglycerin, hydrolyzed sodium hyaluronate, hydroxyacetophenone, panthenol, pantolactone, pentylene glycol, sodium acetylated hyaluronate, sodium chloride, sodium glycolate, sodium hyaluronate, sodium hyaluronate crosspolymer, and PEG-Dimethicone, and dimethyl isosorbide. Other suitable ingredients may be used.

While the foregoing invention has been described with respect to various embodiments and examples, this is solely for the purpose of illustration. It is understood that other embodiments are within the scope of the present invention as expressed in the following claims and their equivalents. Moreover, the above specific examples are to be construed as merely illustrative, and not limitative of the remainder of the disclosure in any way whatsoever. Without further elaboration, the embodiments described are solely for the purpose of illustration. Those skilled in the art will recognize other embodiments may be practiced and modifications and alterations limited only by the spirit and scope of the appended claims.

What is claimed is:

1. A topical composition comprising:
a powder complex including an active component, wherein the powder complex has a pre-defined first dosage; and
a universal activator serum having a pre-defined second dosage, wherein the combination of the powder complex and the universal activator serum generates the topical composition having a third pre-defined dosage and a viscosity of 10-30K centipoise (CPS).

2. The topical composition of claim 1, wherein the universal activator serum has a viscosity of 1 k-5 k CPS.

3. The topical composition of claim 1, wherein the powder complex and universal activator serum are in a 1:2 ratio in the topical composition.

4. The topical composition of claim 1, wherein the active component is stable prior to combination with the universal activator serum.

5. The topical composition of claim 1, wherein the active component is between 0.25-10% of the topical composition.

6. The topical composition of claim 1, wherein the powder complex further comprises:
one or more viscosity controlling agents and one or more moisturizing agents.

7. The topical composition of claim 6, wherein the powder complex further comprises at least one of:
one or more solvents, one or more humectants, one or more absorbents, one or more skin conditioning agents, one or more pigments, one or more aromatic agents, one or more preservatives and one or more pH adjustor agents.

8. The topical composition of claim 1, wherein the universal activator serum comprises one or more viscosity controlling agents and one or more moisturizing agents.

9. The topical composition of claim 1, wherein the active component is at least one of cosmetically and pharmaceutically acceptable for topical application to skin.

10. The topical composition of claim 1, wherein the powder complex has a bulk density in a range of +/−10% of 0.51 g/ml.

11. The topical composition of claim 1, wherein the powder complex and universal activator serum are combined on a mixing surface to form the topical composition.

12. The topical composition of claim 11, wherein the surface is one of skin and a receptacle.

13. The topical composition of claim 1, wherein the powder complex is combined with the universal activator serum for ten seconds or less to form the topical composition.

14. The topical composition of claim 1, wherein the powder complex is at least one of suspended and dissolved in the universal activator serum.

15. The topical composition of claim 9, wherein the topical composition has a pH of 3.5-7.0.

16. The topical composition of claim 1, wherein the active component is one of Vitamin C, a Botanical extract, CoQ10, an Enzyme, Probiotic, and Retinol.

17. A method of forming a topical composition comprising:
providing a source of a powder complex including an active component;
providing a source of a universal activator serum;
dispensing a predefined first dosage of the powder complex onto a surface;
dispensing a pre-defined second dosage of the universal activator serum onto the surface;
combining the dispensed powder complex with the dispensed universal activator serum to form the topical composition having a third pre-defined dosage and a viscosity of 10-30K centipoise (CPS).

18. The method of claim 17, wherein the powder complex has a bulk density in a range of +/−10% of 0.51 g/ml.

19. The method of claim 17, wherein combining the dispensed powder complex with the dispensed universal activator serum further comprises:
continuously mixing the dispensed powder complex with the dispensed universal activator serum for a time of 5 to 10 seconds.

20. The method of claim 17, wherein combining the dispensed powder complex with the dispensed universal activator serum at least one of suspends and dissolves the dispensed powder complex in the universal activator serum.
